(19) United States
(12) Patent Application Publication
(10) Pub. No.: US 2005/0171437 A1
(43) Pub. Date: Aug. 4, 2005

(54) Optical switching system for catheter-based analysis and treatment

(75) Inventor: John Carberry, Talbott, TN (US)

Correspondence Address: PITTS AND BRITTIAN P C, P O BOX 51295, KNOXVILLE, TN, 37950-1295, US

(73) Assignee: Neptec Optical Solutions, Inc., Jefferson City, TN (US)

(21) Appl. No.: 10/757,668

(22) Filed: Jan. 14, 2004

(51) Int. Cl. A61B 6/00; A61B 1/06

(52) U.S. Cl. 600/476; 600/160; 600/473; 600/478

(57) ABSTRACT

An optical switching system for use with catheter-based analysis and treatment. The use of an optical switching system allows one or more interferometric systems to use the same fiber, control of the duty cycle to protect the sensitive optical devices from harmful back-reflections generated by a treatment laser, and switching through several interferometric light sources in order to determine geometry and composition in the path of the catheter.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Field of Invention

This invention pertains to apparatus for switching signals, lambdas, or bandgaps of optical spectrum in a catheter. More particularly, this invention pertains to selectively applying a plurality of optical signals to a optical fiber in a catheter and processing the optical signals returned from the catheter.

2. Description of the Related Art

During the past twenty years, the use of catheters to enter, diagnose, and treat diseases and malfunctions of the blood vessels and other vessels has become commonplace. Catheters are widely employed to deliver stents to occluded blood vessels, as well as to position and deploy balloons to enlarge occluded blood vessels. Also, catheters are used in combination with excimer lasers for treating and removing plaque.

Unfortunately, medical professionals are unable to take advantage of the relatively non-invasive catheter in certain cases. For example, in the case of a totally occluded aortic or other vessel, it is difficult or impossible to safely insert and position a catheter due to the difficulty or impracticability of using X-RAY techniques to position the catheter. In approximately 330,000 cases per year, this results in open-heart surgery, which in addition to a long and painful recovery and high expense, carries significant risks.

Similarly, the usefulness of catheters in treating and removing plaque is often limited. Recent findings indicate that nonstenotic, lipid rich coronary plaques, also called “vulnerable plaques” or “biological hot plaques” are exceptionally likely to cause the vast majority of fatal heart attacks. In other words, the majority of the approximately 1,300,000 heart attacks that will occur this year are caused by a soft plaque, for which there is not currently available a viable tool for identifying, diagnosing, or treating. While catheter-based excimer lasers have been proven to be effective at treating and removing soft plaques, their use has been limited by the practitioner's inability to see and control the position of the catheter before, during, and after using the excimer laser.

Various tests exist for identifying persons at risk of myocardial infarction. These persons are candidates for further evaluation and treatment. In such a case, an ideal treatment and system would allow for the use of multiple devices within a single catheter, therefore allowing several functions, some complementary, over the course of a single catheter insertion procedure, which would allow: a) the use of an interferometer capable of navigating the catheter through the blood vessels to allow the catheter to be moved through total occlusions as well as through the twists and turns of the blood vessels; b) the use of an interferometer that could use multiple wavelengths to differentiate among various materials in the optical path, including vulnerable plaque, calcified plaque, arterial walls, etc.; and c) the intermittent use of an excimer or other laser to ablate, vaporize, or otherwise destroy the plaque in the path of the catheter.

There are three primary instruments routinely used in catheter insertion procedures. First, Michelson interferometers of various types are used to differentiate between plaque and arterial walls, and to do so with physical resolution in the range of 10 microns. Michelson interferometers provide the ability to see and navigate through a total occlusion. Second, Diffuse Reflectance Near Infrared Spectroscopy (DRNIRS), often with regard to multiple wavelengths, is effective at differentiating and identifying a wide variety of substances, including hundreds of plasma constituents, such as glucose, calcified plaque, vulnerable plaque, total protein, human metalloproteins, creatinine, uric acid, triglycerides, uric acid, urea, etc. DRNIRS interferometry provides the capability to detect and determine materials without actually contacting or touching them. The substances are distinguished by the characteristic absorption and reflectance of specific wavelengths of light, typically between 300 and 2200 nanometers. Third, excimer lasers typically use a very short pulse, less than 1 microsecond, normally about 100 nanoseconds, and could be operated together with both types of interferometry in duty cycles as high as hundreds of hertz.

Other devices for evaluating and treating arterial disease are known to those skilled in art. As with all optical devices, it is generally known to use either a single fiber or a bundle of fibers to transmit one or more optical signals. Often these devices are intended to improve the resolution and/or information available using the known navigation and diagnostic techniques and focus on improving a single technique. Examples of such uses are described in the following U.S. patents. U.S. Pat. No. 5,217,456, issued to Narciso, Jr., discloses a catheter for ablation of a lesion. The rotating catheter has a bundle of optical fibers that are used to make fluorescence measurements to identify the radial position of the lesion. U.S. Pat. No. 6,384,915, issued to Everett, et al., and U.S. Pat. No. 6,175,669, issued to Colston, et al., disclose the use of a multiplexed reflectometer for performing Michelson interferometry. Both patents describe a system including a optical fiber set contained within the catheter. The optical fibers are connected to the illumination source via an optical switch, which sequentially cycles the output of the source through the optical fiber set to diagnose consecutive spatially-distinct regions of a lumen. U.S. Pat. No. 6,463,313, issued to Winston, et al., describes a device having dual Michelson interferometers. The outputs are combined to produce a composite image thereby providing more complete information to the medical professional. U.S. Pat. No. 6,501,551, issued to Tearney, et al., discloses the combination of two sources of differing wavelengths using wavelength division multiplexing. The combined signal is injected into a single optical fiber in the catheter. The reflections are separated by wavelengths and guided to separate detectors associated with a particular wavelength.

Devices combining some navigation or diagnostic element, such as a Michelson interferometer, with a treatment element, such as a excimer laser, are known to those skilled in the art. These devices are represented by the angioplasty systems such as the those described in U.S. Pat. No. 5,275,594, issued to Baker, et al. and in U.S. Pat. No. 6,463,313, Winston, et al. Both Baker, et al., and Winston, et al., disclose systems that use feedback from the diagnostic element to control the operation of the treatment element. U.S. Pat. No. 6,389,307, issued to Abela, discloses a system having a lower power diagnostic laser and a high power treatment laser coupled to the same optical fiber. The operator activates the desired laser, preferably one at a time, to achieve a desired function.

An optical switching system for use with a catheter-based analysis and treatment instrument that facilitates a procedure that combines navigation, identification, and correction within the domain of insertion and operation during a single catheter experience or procedure would offer dramatic benefits to save lives and preclude coronary events. This procedure would be an effective, efficient, and safe method for treating a very dangerous condition, especially when compared to the options of performing no procedure or performing a bypass surgery.

### BRIEF SUMMARY OF THE INVENTION

An apparatus and method for treatment of the arteries of the heart using optical switches to allow safe navigation of blood vessels with a catheter through the use of one or more interferometer systems and intermittent or concurrent treatment through the use of a treatment laser, precise insertion of a stent to cover the hot plaque, or other tool. The apparatus and method allows differentiation among arterial walls, calcified plaque, vulnerable plaque, such as Biological Hot Plaque, thin capped fibrous atheromas (TCFAs), and other forms and substance in blood vessels. The device and method is useful in the treatment of Atherosclerosis, Arteriosclerosis, and Thrombosis, the performance of Hemodialysis Access Maintenance, and the insertion of Trans jugular Intrahepatic Portosystemic Shunts.

The apparatus allows multiple optical sources to be switched into one or more optical fibers in the catheter. The return signal from the catheter is switched between multiple optical detectors, such as an interferometer, a spectrum analyzer, and a reflectometer. The use of optical switches allows the use of one or more interferometric systems in the same fiber, as well as using the switches to control a duty cycle that protects the optical source and detectors and other vulnerable or sensitive optical devices from harmful back reflections generated by the short but powerful pulses of an excimer, or other, laser or light source, or in the case that such devices are not in danger of being harmed by back reflection, switching through several interferometric light sources in order to determine geometry and composition in the path of the catheter.

The use of an optical switch provides the capability to sample multiple lambdas and/or bandwidth spectra through a fiber and from the loci of a single fiber end in the catheter into the loci of a single point on an artery wall quick enough to safely assure that all the sampling of lambdas or bandwidth spectra occurred in the same loci in the artery allowing an inference as to the composition at that loci on the artery wall, allowing to differentiate among artery wall, calcified plaque, hot plaque and other materials.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 illustrates one embodiment of an optical switching system for use with catheter-based analysis and treatment;

FIG. 2 illustrates one embodiment of the catheter adapted for use with the present invention;

FIG. 3 illustrates the catheter of the present invention in the environment of an artery;

FIG. 4 illustrates an alternate embodiment of the optical switching system for use with catheter-based analysis and treatment incorporating a treatment laser;

FIG. 5 illustrates an alternate embodiment of the optical switching system for use with catheter-based analysis and treatment adapted for using external optical sources and detectors;

FIG. 6 illustrates an alternate embodiment of the optical switching system for use with catheter-based analysis and treatment adapted for using external optical sources and detectors and incorporating a treatment laser;

FIG. 7 is a flow chart of one method of sequencing the source switch and detector switch in relation to the catheter switch; and

FIG. 8 is a flow chart of an alternate method of sequencing the source switch and detector switch in relation to the catheter switch.

### DETAILED DESCRIPTION OF THE INVENTION

An optical switching system for use with catheter-based analysis and treatment, or optical switching system, is shown and described. The use of an optical switching system allows the use of one or more interferometric systems in the same fiber, as well as using the optical switching system to control a duty cycle that protects the optical source and detectors and other vulnerable or sensitive optical devices from harmful back-reflections generated by the short pulses of a high-power light source, such as an excimer laser, or in the case that such devices are not in danger of being harmed by back reflection, switching through several interferometric light sources in order to determine geometry and composition in the path of the catheter.

The use of optical switches greatly aids in safely constructing and using a device for locating, identifying, and removing a blockage. First, an optical switch allows the use of multiple wavelengths and the insertion of these into one or more optical fibers by rapidly switching among the available wavelengths. It is important to note that while various types of interferometry may often be performed on a single type of fiber, for the most part they cannot be operated at the same time as they would interfere with the functionality and resolution of the various interferometers. For this reason, in the case where multiple interferometers are useful, the optical switch permits one or more interferometers to operate through the same optical fiber set. For example, the procedure can use Michelson interferometry for navigating through a total occlusion and use Diffuse Reflectance Near Infrared Spectroscopy (DRNIRS) for differentiating between blood, water, vulnerable plaque, calcified plaque, and other objects. Similarly, when using identifying interferometry, various wavelengths are required to identify different materials, such as calcium rich plaque, vulnerable plaque, blood, water, arterial walls, etc. Again, the optical switch allows the necessary wavelengths to be switched through the optical fibers. Second, an optical switch provides the ability to return the reflectances from the end of the catheter to multiple interferometry devices. Third, an optical switch makes it possible to break the optical connection to both optical sources and optical detectors during the use and duty cycle of a high-power laser. By taking the optical sources and optical detectors off-line protects them from harmful and potentially destructive back reflections, to which such devices are exceptionally vulnerable.

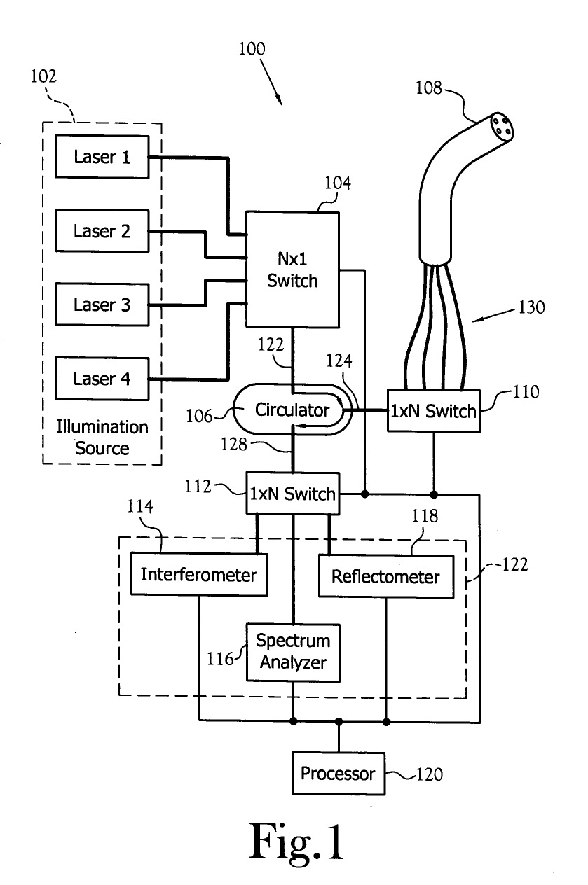

FIG. 1 illustrates one embodiment of a catheter-based analysis and treatment instrument incorporating an optical switching system in a according to the present invention. The medical apparatus includes a multi-wavelength illumination source 102, often a bank of low coherence lasers, that is optically connected to. a first optical switch 104. The illumination source 102 is generally any coherent light source that can be used for medical imaging and that can be properly carried by an optical fiber. In one embodiment, each of the lasers in the illumination source 102 has a unique wavelength and generates a coherent light beam that is useful for navigation of a lumen and/or differentiation or identification of objects within the lumen. The first optical switch 104 allows selection of one of the lasers from the bank 102 to be directed through a catheter 108. The first port 122 of a circulator 106, which is optically connected to the first optical switch 104, redirects the selected laser beam through a second port 124 into a second optical switch 110. The second optical switch 110, which is optically connected to the circulator 106, sequentially cycles the selected laser beam through a plurality of optical fibers 130 routed through the catheter 108. The reflections of the laser beam from the catheter 108 are fed back into the circulator 106 through the second port 124 and redirected through the third port 128 of the circulator 106 into a third optical switch 112. The third optical switch 112 connects the reflections of the laser beams to various optical detectors 122. In the illustrated embodiment, the third optical switch 112 is connected to an interferometer 114, a spectrum analyzer 116, and a reflectometer 118. A processing device 120 controls the switching operations for the first optical switch 104, the second optical switch 110, and the third optical switch 112. In addition, the processing device 120 communicates with the optical detectors 122.

As illustrated and described herein, the optical circulator 106 passes signals between successive ports in one direction. However, those skilled in the art will recognize that single direction signal paths can be achieved using other devices including optical switches. The bank of lasers 102 is presumed to have multiple sources; however, those skilled in the art will recognize that a single tunable laser or other tunable source capable of generating the desired wavelengths could be used. In such an arrangement, the single source subsumes the functions of the multiple sources and the first optical switch without departing from the spirit and scope of the present invention. Similarly, the optical detectors 122 is illustrated as including multiple devices performing differing functions. Those skilled in the art will recognize that the optical detectors may include only a single analysis device or single multi-function analysis device and would not require the third optical switch. In either event, such a substitution could easily be warranted by advances in the illumination source or the optical detectors or may merely reflect a medical apparatus performing fewer functions than the illustrated embodiment.

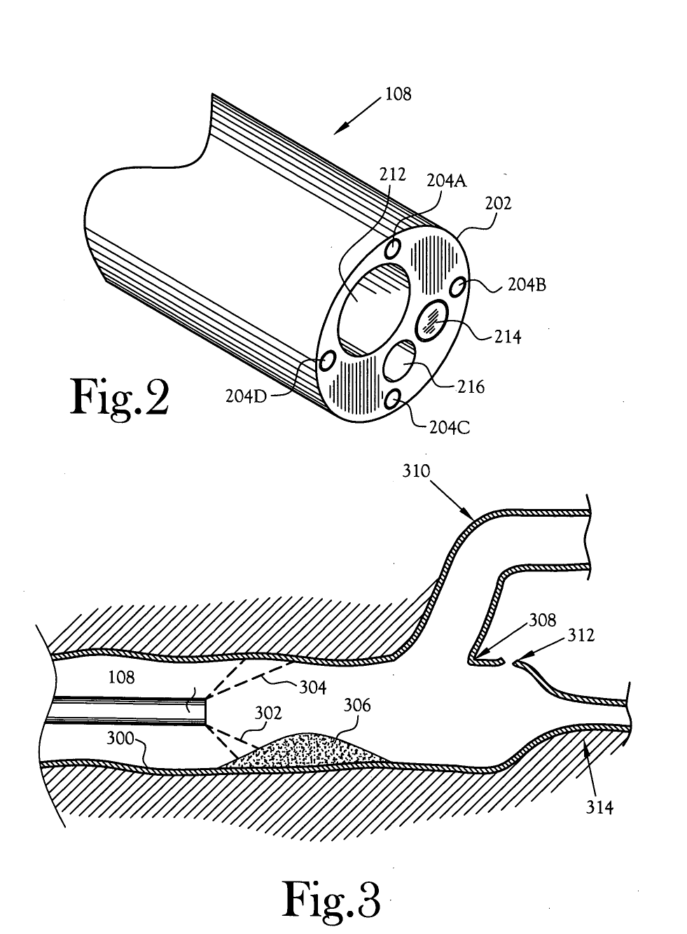

FIG. 2 illustrates the construction of the catheter 108 in greater detail. The primary tube 202 of the catheter 108 defines a number of channels that carry or remove various fluids or route or carry other cables, wires, and implements. In the illustrated embodiment, the catheter 108 carries four optical fibers 204A, 204B, 204C, 204D arranged at cardinal points in the cross-section of the catheter 108. The catheter 108 defines a large channel 212 through which various implements, such as balloons or stents, can be inserted and manipulated. The catheter 108 also carries a guide wire 214. In addition, the catheter 108 defines a channel through which various fluids can be introduced and removed, for example, to inflate an angioplasty balloon. Accordingly, the catheter of the present invention incorporates multiple optical fibers fed by an optical switch with other medically necessary and/or useful features; however, those skilled in the art will recognize that configuration and features of the catheter depend upon the usage for which the catheter is designed.

Those skilled in the art will recognize that the number of optical fibers depends upon the desired field of vision and the image processing occurring at the analysis device and, therefore, that number can be varied without departing from the scope and spirit of the present invention. Similarly, the arrangement of the optical fibers depends both upon number and the desired field of vision. Typical, the optical fibers will be equidistantly spaced around the perimeter of the primary tube to provide the most complete field of vision; however, those skilled in the art will recognize other arrangements may be used without departing from the scope and spirit of the present invention.

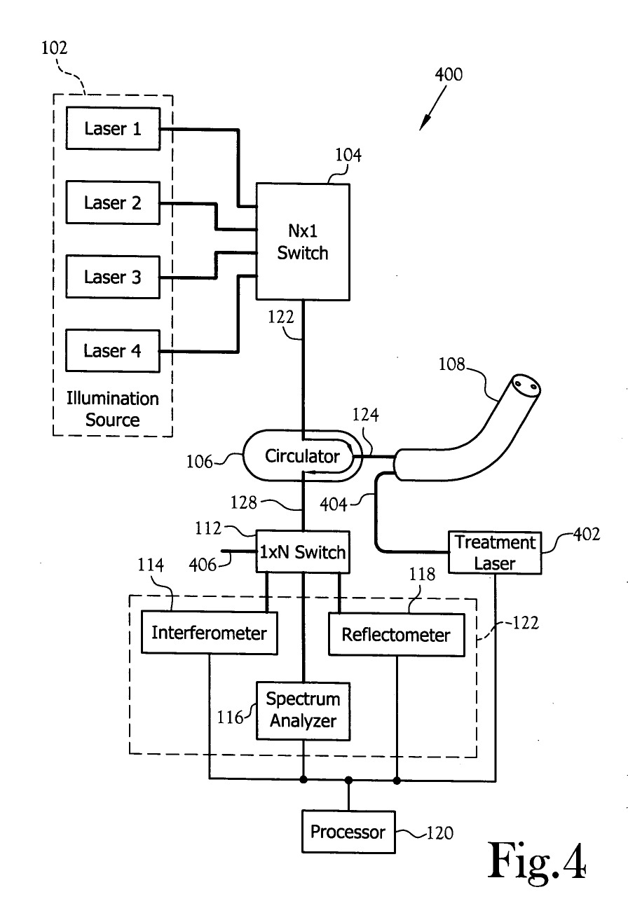

FIG. 3 is a cross-section showing the catheter 108 navigating through a blood vessel 300. The dashed cones represent the upper field of view 302 and the lower field of view 304. The left and right side fields of view are not depicted. In the illustrated embodiment, the blood vessel 300 includes a variety of objects which require navigation or identification. The objects include a bump 306, such as a plaque deposit, a bifurcation 308 of the blood vessel, a turn 310 in the blood vessel, an aortic dissection 312 (or other similar damage to the blood vessel), and a closure or narrowing 314 of the blood vessel.

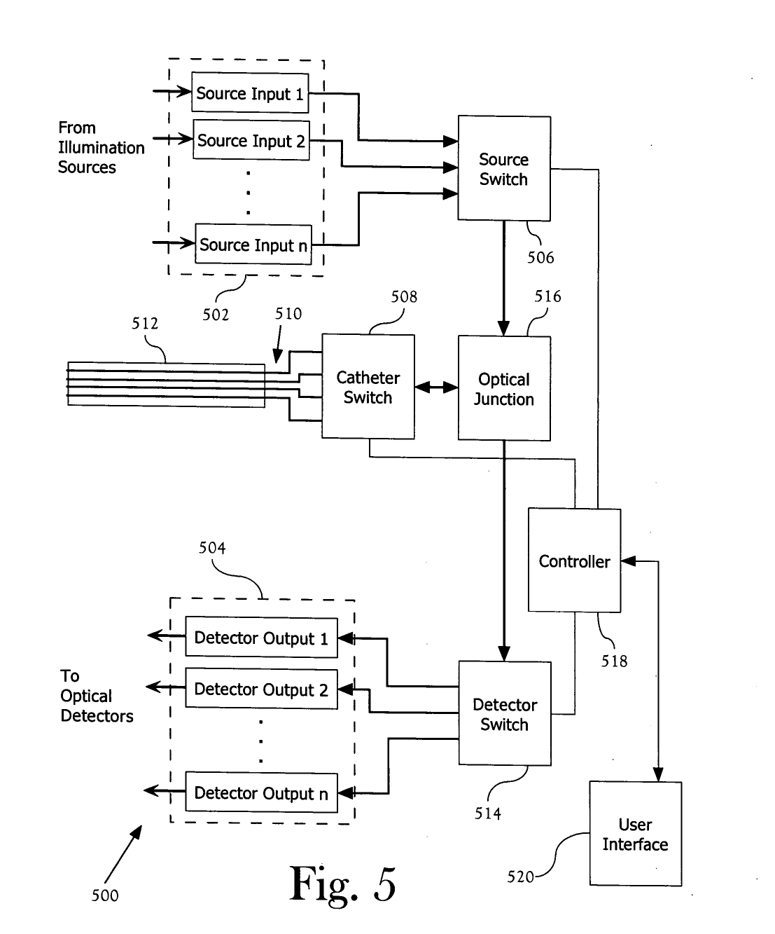

FIG. 4 illustrates an alternate embodiment of a medical apparatus 400 incorporating an optical switching system in a catheter-based analysis and treatment instrument according to the present invention. The medical apparatus 400 includes a treatment laser 402, such as an excimer laser or similar laser, used for evaporation or ablation of an arterial blockage, such as a plaque deposit. A separate optical fiber 404 in optical communication with the treatment laser 402 runs through the catheter 408. The medical apparatus 400 also includes a shunt 406 that is connected to the optical path during the operation of the treatment laser 402. The shunt 406 is a dead-end optical path where higher power reflectances from the treatment laser 402, which return through the optical fiber 124, are routed to prevent damage to the sensitive interferometry devices 122.

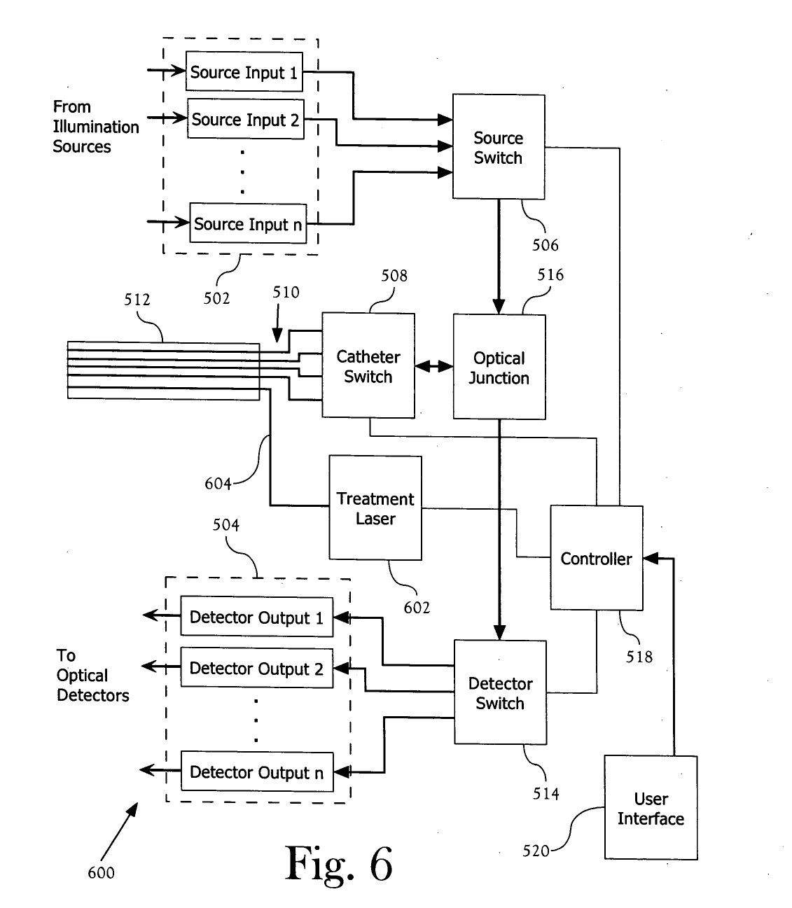

FIG. 5 illustrates yet another embodiment of the medical apparatus 500 adapted for optical navigation and optical identification in conjunction with non-optical treatments, such as stent insertion or angioplasty. This embodiment of the medical apparatus 500 includes a plurality of input ports 502 for receiving optical signals from external optical sources, and a plurality of output ports 504 for transmitting optical signals to external optical detectors (not shown). The input ports 502 are routed through an optical switch 506. The input port optical switch 506 is optically connected to another optical switch 508 associated with a group of optical fibers 510 carried by a catheter 512. The catheter optical switch 508 is also optically connected to a third optical switch 514 associated with the plurality of output ports 504.

The three optical switches 506, 508, 514 are interfaced by an optical junction 516. The primary function of the optical junction 516 is to route the optical signals to the appropriate destination. This generally means that source signals are routed into the catheter and the reflectances returning from the catheter are routed to the output ports. A secondary function of the optical junction 516 is to prevent optical signals from traveling to undesirable destinations. This generally means that the reflectances are prevented from reaching the input ports 502 and the source signals are prevented from directly reaching the output ports 504. These two functions are realized by implementing the optical junction with an optical circulator; however those skilled in the art will recognize that the optical junction can be built from combinations of other optical components including splitters, multiplexers, demultiplexers, and switches without departing from the scope and spirit of the present invention.

A controller 518 coordinates the operation of the three optical switches 506, 508, 514 so that the reflectances of an input signal of a certain type or wavelength are directed to the appropriate detector for analysis. This is facilitated by software routines processed by the controller 518 and commands received from an optional user interface 520. If required, the optical junction can also be placed under the control of the controller 518.

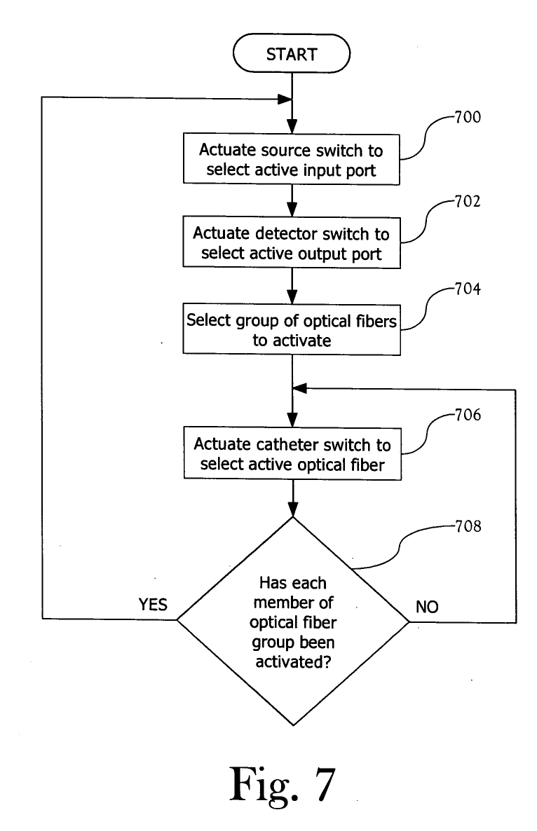

FIG. 6 illustrates still another embodiment of the medical apparatus 500 adapted for optical navigation, identification and treatment. This embodiment expands upon that shown in FIG. 5 with the inclusion of a treatment laser 602 and another optical fiber 604 in the catheter 512 for carrying the high power bursts of the treatment laser 602. The operation of the treatment laser 602 is coordinated in the system by the controller 520. Generally, during the operation of the treatment laser 602, any or all of the other optical switches are moved to a safe position to optically isolate the optical sources and detectors from potentially harmful back-reflections of the treatment laser 602. The safe position could be any position if the optical circulator provides optical isolation or can be a special position which connects the optical fibers 510 of the catheter 512 to optical dead-ends.

It should be noted that while the illustrated embodiments of FIGS. 1, 4, 5, and 6 show all three optical used together, the use of a single source switch, a single detector switch, and the various sub-combinations of the three switches are also contemplated by the present invention.

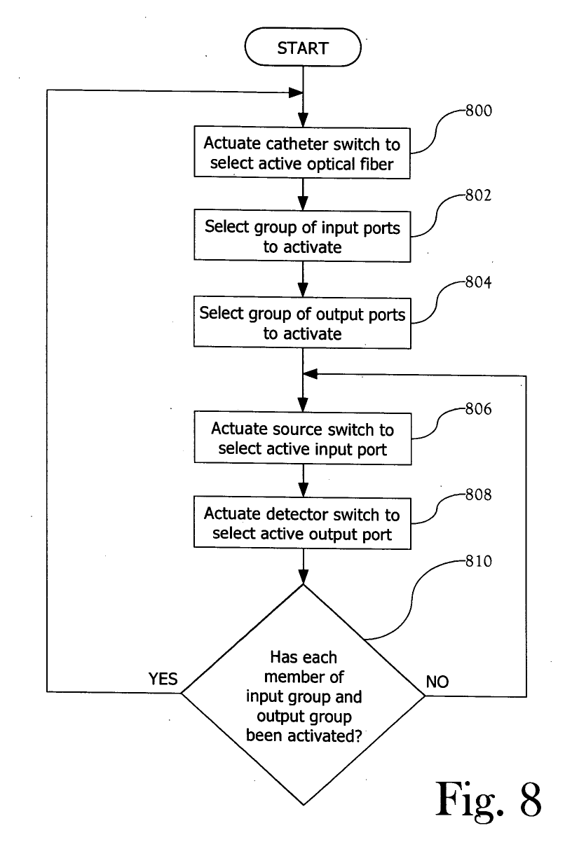

Another feature of the present invention is the ability to control the routing of the optical sources through the catheter to obtain a full picture of the lumen. By sending the signal from each optical source through a selected group of the optical fibers in the catheter a more accurate picture of the lumen is obtained. FIG. 7 is flow chart of the sequencing of the optical sources relative to the optical fibers in the catheter. First, the controller actuates the source switch 700 making a selected input port active so that signals from a desired source can be used. The controller also actuates the detector switch 704 making a selected output port active so that reflectances from the input signals are routed to the desired detector. A group of optical fibers is selected 706. This selection can be static, i.e., the same every time, or exhibit variability based upon detected conditions or user control. It is common for the group to include each optical fiber; however, subsets of the optical fibers can be selected. Next, the controller actuates the catheter switch to select the active optical fiber 706. This continues until each optical fiber in the group has been used 708. Those skilled in the art will recognize the activation sequence of the optical fibers can be varied without departing from the scope and spirit of the present invention.

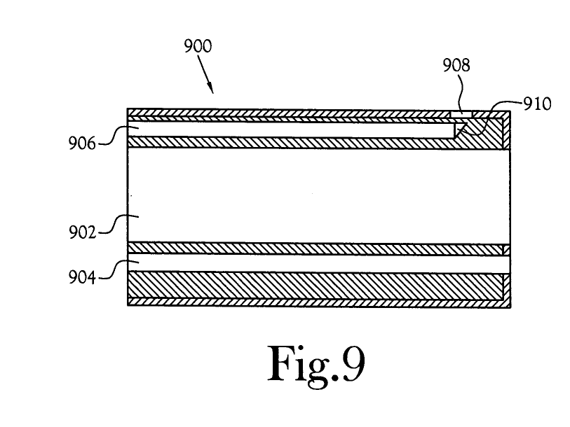

FIG. 8 is a flowchart of a variation on the sequencing function shown and described in reference to FIG. 7. In this variation, the active optical fiber in the catheter remains constant while the input ports and the corresponding output ports are rotated. First, the controller selects the active optical fiber in the catheter 800. Next, the group of input ports associated with the desired sequence of input sources is selected 802. This is followed by the selection the group of output ports associated with the desired optical detectors 804. Note that the input sources and detectors need not follow a one-to-one correspondence, as the reflectances from a single input source may be used by multiple optical detectors. The controller actuates the source switch to cycle through the selected group of input ports 806. The controller also actuates the detector switch to cycle through the selected group of output ports 808. The source switch and detector switch actuation continues until all selections of the input port group and the output port group have been made active 810.

FIG. 9 illustrates a cross-section of an alternate embodiment of the catheter 900 utilizing a single optical fiber 906. Some of the basic features of the catheter 900 are visible in FIG. 9. The catheter 900 defines a large channel 902 through which various implements, such as balloons or stents, can be inserted and manipulated. The catheter 900 also carries a guide wire 904. The optical fiber 906 is disposed proximate to the perimeter of the catheter 900. In the illustrated embodiment, the optical fiber 906 has a 200 micron core, although, those skilled in the art will recognize that other core sizes can be used without departing from the scope and spirit of the present invention. It is desirable to minimize the amount of blood between the end of the optical fiber and the point of interest in the artery, i.e., the arterial wall and the artifacts thereon. Accordingly, in the illustrated embodiment, the optical fiber includes a mirrored surface 910 disposed at an angle approximating 45 degrees. The mirrored surface 910 causes the lambas to exit the optical fiber 906 at a roughly 90-degree angle through a window 908 in the wall of the catheter 908 and intersect the arterial wall. By rotating the catheter 906, a full 360-degree view is obtained. Those skilled in the art will recognize that any number of optical fibers can be used without departing from the scope and spirit of the present invention.

The usefulness of the information obtained is largely dependent upon the acquisition speed of the information. A rapid acquisition speed allows both navigation and identification information to be obtained about the same location in the artery. If the acquisition speed is to low, the navigation information and the identification information are not associated with the same location within the artery and do not provide a complete picture. Obviously, the switching speed is dependent upon the forward movement speed and/or the rotational speed of the catheter and the number of wavelengths required to obtain a complete picture. The present inventor has found that a switching speed in the range of 30 to 50 milliseconds provides a sufficient data acquisition speed for most applications, although other switching time ranges are acceptable. The optical switching system of the present invention is capable of operating at the necessary switching speed to obtain useful information.

Certain characteristics of the optical switching system are useful in providing an efficient implementation; however, those skilled in the art will recognize that these characteristics are intended to be exemplary and not limiting. In various embodiments, the optical switching system latches exhibits low optical loss, nominally less than 1 dB, and low port to port variability, nominally less than 0.5 dB. The optical switching system latches in all positions, making the switch stable, resistant to shock and vibration and unintentional switching. The optical switching system exhibits temperature Independent operation with regard to optical performance. The optical switching system exhibits low polarization dependent loss, nominally less than 0.2 dB. The optical switching system exhibits a switching time quicker than 100 milliseconds.

From the foregoing description, it will be recognized by those skilled in the art that a device and method for safely navigating blood vessels using a catheter has been provided. The device and method uses an optical switch to control the inputs and outputs of optical fibers set in a catheter. The device can differentiate among arterial walls, calcified plaque, vulnerable plaque (biological hot plaque and thin capped fibrous atheromas), and other forms and substances in blood vessels. The device is useful in the treatment of the arteries of the heart, Atherosclerosis, Arteriosclerosis, Thrombosis, for the performance of Hemodialysis Access Maintenance, and for the insertion of Transjugular Intrahepatic Portosystemic Shunts. In addition, the device provides for the intermittent or concurrent use of a treatment laser, such as an excimer laser, or other treatment tool, such as a stent or an angioplasty balloon, in conjunction with one or more interferometer systems and devices by use of optical switches.

While the present invention has been illustrated by description of several embodiments and while the illustrative embodiments have been described in detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

1. A medical apparatus for safely navigating a lumen using a catheter, which can also differentiate between various objects found within the lumen, and for treating various conditions existing within the lumen, said medical apparatus comprising:
a catheter;
a plurality of diagnostic optical fibers carried by said catheter;
a plurality of optical source inputs;
a plurality of optical detector outputs;
an optical junction;
a first optical switch in optical communication between said plurality of optical source inputs and said optical junction, said first switch selectively optically connecting one of said plurality of optical source inputs to said optical junction;
a second optical switch in optical communication between said plurality of optical detector outputs and said optical junction, said second switch optically connecting one of said plurality of optical detector outputs to said optical junction so as to receive a reflectance introduced in said plurality of diagnostic optical fibers subsequent to activation of one of said plurality of optical source inputs; and
a third optical switch in optical communication between said optical junction and said plurality of diagnostic optical fibers, said third switch selectively optically connecting said optical junction to one of said plurality of diagnostic optical fibers.

2. The medical apparatus of claim 1 further comprising a controller providing position awareness of and switching control over said first optical switch, said second optical switch, and said third optical switch.

3. The medical apparatus of claim 1 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said first optical switch to connect a selected one of said first optical switch plurality of inputs to said first optical switch output;
cycling said third optical switch such that said third optical switch input is sequentially connected to each of a selected group of said third optical switch plurality of outputs.

4. The medical apparatus of claim 1 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said third optical switch to connect said third optical switch input to a selected one of said third optical switch plurality of outputs;
cycling said first optical switch such that each of a selected group of said first optical switch plurality of inputs are sequentially connected to said first optical switch output.

5. The medical apparatus of claim 1 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said second optical switch such that said second optical switch input is connected to a selected one of said second optical switch plurality of outputs;
cycling said third optical switch such that said third optical switch input is sequentially connected to each of a selected group of said third optical switch plurality of outputs.

6. The medical apparatus of claim 1 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said third optical switch to connect said third optical switch input to a selected one of said third optical switch plurality of outputs;
cycling said second optical switch such that said second optical switch input is sequentially connected to each of a selected group of said second optical switch plurality of outputs.

7. The medical apparatus of claim 1 wherein said optical junction optically isolates said plurality of optical source inputs from said reflections.

8. The medical apparatus of claim 1 wherein said optical junction is an optical circulator configured to optically connect said plurality of optical source inputs with said plurality of diagnostic optical fibers in a first direction and optically isolate said plurality of optical source inputs from said plurality of diagnostic optical fibers in an opposite direction, to optically connect said plurality of optical detector outputs with said plurality of diagnostic fibers, and to optically isolate said plurality of optical detector outputs from said plurality of optical source inputs.

9. The medical apparatus of claim 1 further comprising:
a treatment optical fiber carried by said catheter; and
a treatment laser optically connected to said treatment optical fiber.

10. The medical apparatus of claim 1 further comprising:
a treatment optical fiber carried by said catheter; and
a treatment laser optically connected to said treatment optical fiber, wherein said first optical switch optically isolates said plurality of optical source inputs from said plurality of diagnostic optical fibers during operation of said treatment laser.

11. The medical apparatus of claim 1 further comprising:
a treatment optical fiber carried by said catheter; and
a treatment laser optically connected to said treatment optical fiber, wherein said second optical switch optically isolates said plurality of optical detector outputs from said plurality of diagnostic optical fibers during operation of said treatment laser.

12. The medical apparatus of claim 1 further comprising a conduit defined by said catheter, said at conduit adapted for a procedure selected from the group consisting of fluid removal, angioplasty balloon insertion, angioplasty balloon inflation, and stent insertion.

13. The medical apparatus of claim 1 further comprising a plurality of sources each producing light of a selected wavelength, each of said plurality of sources connected to one of said plurality of optical source inputs.

14. The medical apparatus of claim 1 further comprising a plurality of detectors each responsive to light of a selected wavelength, each of said plurality of detectors connected to one of said plurality of optical detector outputs.

15. A medical apparatus for safely navigating a lumen using a catheter, which can also differentiate between various objects found within the lumen, and for treating various conditions existing within the lumen, said medical apparatus comprising:
a catheter;
a diagnostic optical fiber carried by said catheter;
a plurality of optical source inputs;
an optical junction in optical communication with said diagnostic optical fiber;
an optical switch in optical communication between said plurality of optical source inputs and said optical junction, said optical switch optically connecting a selected one of said plurality of optical source inputs to said optical junction; and
an optical detector output responsive to light of a selected wavelength, said optical detector output in optical communication with said optical junction so as to receive a reflectance introduced in said diagnostic optical fiber subsequent to activation of one of said plurality of optical source inputs.

16. The medical apparatus of claim 15 further comprising a controller providing position awareness of and switching control over said optical switch.

17. The medical apparatus of claim 15 wherein said plurality of optical source inputs is optically isolated from said reflections.

18. The medical apparatus of claim 15 wherein said optical junction is an optical circulator configured to optically connect said plurality of optical source inputs with said diagnostic optical fiber in a first direction and optically isolate said plurality of optical source inputs from said diagnostic optical fiber in an opposite direction, to optically connect said optical detector output with said diagnostic optical fiber, and to optically isolate said optical detector output from said plurality of optical source inputs.

19. The medical apparatus of claim 15 further comprising:
a treatment optical fiber carried by said catheter; and
a treatment laser optically connected to said treatment optical fiber

20. The medical apparatus of claim 15 further comprising:
a treatment optical fiber carried by said catheter; and
a treatment laser optically connected to said treatment optical fiber, wherein said first optical switch optically isolates said plurality of optical source inputs from said diagnostic optical fiber during operation of said treatment laser.

21. The medical apparatus of claim 15 further comprising a conduit defined by said catheter, said at conduit adapted for a procedure selected from the group consisting of fluid removal, angioplasty balloon insertion, angioplasty balloon inflation, and stent insertion.

22. The medical apparatus of claim 15 further comprising a plurality of sources each producing light of a selected wavelength, each of said plurality of sources connected to one of said plurality of optical source inputs.

23. The medical apparatus of claim 15 wherein said diagnostic optical fiber ends in a mirror disposed at a substantially 45 degree angle.

24. A medical apparatus for safely navigating a lumen using a catheter, which can also differentiate between various objects found within the lumen, and for treating various conditions existing within the lumen, said medical apparatus comprising:
a catheter;
a diagnostic optical fiber carried by said catheter;
an optical source input producing light having a selected wavelength;
a plurality of optical detector outputs;
an optical junction optically connecting said optical source input and said diagnostic optical fiber; and
an optical switch in optical communication between said plurality of optical detector outputs and said optical junction, said optical switch optically connecting one of said plurality of optical detector outputs to said optical junction so as to receive reflectances introduced in said diagnostic optical fiber subsequent to activation of said optical source input.

25. The medical apparatus of claim 24 further comprising a controller providing position awareness of and switching control over said optical switch.

26. The medical apparatus of claim 24 wherein said optical source input is optically isolated from said reflectances.

27. The medical apparatus of claim 24 wherein said optical junction is an optical circulator configured to optically connect said optical source input with said diagnostic optical fiber in a first direction and optically isolate said optical source input from said diagnostic optical fiber in an opposite direction, to optically connect said plurality of optical detector outputs with said diagnostic optical fiber, and to optically isolate said plurality of optical detector outputs from said optical source input.

28. The medical apparatus of claim 24 further comprising:
a treatment optical fiber carried by said catheter; and
a treatment laser optically connected to said treatment optical fiber.

29. The medical apparatus of claim 24 further comprising:
a treatment optical fiber carried by said catheter; and
a treatment laser optically connected to said treatment optical fiber, wherein said optical switch optically isolates said plurality of optical detector outputs from said diagnostic optical fiber during operation of said treatment laser.

30. The medical apparatus of claim 24 further comprising:
a treatment optical fiber carried by said catheter;
a treatment laser optically connected to said treatment optical fiber; and
an optical dead-end in communication with said optical switch, said optical switch optically connecting said optical dead-end and said optical junction during operation of said treatment laser.

31. The medical apparatus of claim 24 further comprising a conduit defined by said catheter, said at conduit adapted for a procedure selected from the group consisting of fluid removal, angioplasty balloon insertion, angioplasty balloon inflation, and stent insertion.

32. The medical apparatus of claim 24 further comprising a plurality of detectors each responsive to light of a selected wavelength, each of said plurality of detectors connected to one of said plurality of optical detector outputs.

33. The medical apparatus of claim 24 wherein said diagnostic optical fiber ends in a mirror disposed at a substantially 45 degree angle.

34. A medical apparatus for safely navigating a lumen using a catheter, which can also differentiate between various objects found within the lumen, and for treating various conditions existing within the lumen, said medical apparatus comprising:
a catheter;
a diagnostic optical fiber carried by said catheter;
a plurality of optical source inputs;
a plurality of optical detector outputs;
an optical junction in optical communication with said diagnostic optical fiber;
a first optical switch in optical communication between said plurality of optical source inputs and said optical junction, said first optical switch optically connecting a selected one of said plurality of optical source inputs to said optical junction; and
a second optical switch in optical communication between said plurality of optical detector outputs and said optical junction, said second optical switch optically connecting one of said plurality of optical detector outputs to said optical junction so as to receive reflectances introduced in said diagnostic optical fiber subsequent to activation of one of said plurality of optical source inputs.

35. The medical apparatus of claim 34 further comprising a controller providing position awareness of and switching control over said first optical switch and said second optical switch.

36. The medical apparatus of claim 34 wherein said plurality of optical source inputs is optically isolated from said reflections.

37. The medical apparatus of claim 34 wherein said optical junction is an optical circulator configured to optically connect said plurality of optical source inputs with said diagnostic optical fiber in a first direction and optically isolate said plurality of optical source inputs from said diagnostic optical fiber in an opposite direction, to optically connect said plurality of optical detector outputs with said diagnostic optical fiber, and to optically isolate said plurality of optical detector outputs from said plurality of optical source inputs.

38. The medical apparatus of claim 34 further comprising:
a treatment optical fiber carried by said catheter; and
a treatment laser optically connected to said treatment optical fiber.

39. The medical apparatus of claim 34 further comprising:
a treatment optical fiber carried by said catheter; and
a treatment laser optically connected to said treatment optical fiber, wherein said first optical switch optically isolates said plurality of optical source inputs from said diagnostic optical fiber during operation of said treatment laser.

40. The medical apparatus of claim 34 further comprising:
a treatment optical fiber carried by said catheter; and
a treatment laser optically connected to said treatment optical fiber, wherein said second optical switch optically isolates said plurality of optical detector outputs from said diagnostic optical fiber during operation of said treatment laser.

41. The medical apparatus of claim 34 further comprising:
a treatment optical fiber carried by said catheter; and
a treatment laser optically connected to said treatment optical fiber; and
an optical dead-end in communication with said second optical switch, said second optical switch optically connecting said optical dead-end and said optical junction during operation of said treatment laser.

42. The medical apparatus of claim 34 further comprising a conduit defined by said catheter, said at conduit adapted for a procedure selected from the group consisting of fluid removal, angioplasty balloon insertion, angioplasty balloon inflation, and stent insertion.

43. The medical apparatus of claim 34 further comprising a plurality of sources each producing light of a selected wavelength, each of said plurality of sources connected to one of said plurality of optical source inputs.

44. The medical apparatus of claim 34 further comprising a plurality of detectors each responsive to light of a selected wavelength, each of said plurality of detectors connected to one of said plurality of optical detector outputs.

45. The medical apparatus of claim 34 wherein said diagnostic optical fiber ends in a mirror disposed at a substantially 45 degree angle.

46. A medical apparatus for safely navigating a lumen using a catheter, which can also differentiate between various objects found within the lumen, and for treating various conditions existing within the lumen, said medical apparatus comprising:
a catheter;
a plurality of diagnostic optical fibers carried by said catheter;
a plurality of optical source inputs;
an optical junction;
a first optical switch in optical communication between said plurality of optical source inputs and said optical junction, said first optical switch optically connecting a selected one of said plurality of optical source inputs to said optical junction;
a second optical switch in optical communication between said optical junction and said plurality of diagnostic optical fibers, said second optical switch optically connecting a selected one of said plurality of diagnostic optical fibers to said optical junction; and
an optical detector output responsive to light of a selected wavelength, said optical detector output in optical communication with said optical junction so as to receive a reflectance introduced in said plurality of diagnostic optical fibers subsequent to activation of one of said plurality of optical source inputs.

47. The medical apparatus of claim 46 further comprising a controller providing position awareness of and switching control over said first optical switch and said second optical switch.

48. The medical apparatus of claim 46 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said first optical switch to connect a selected one of said first optical switch plurality of inputs to said first optical switch output;
cycling said third optical switch such that said third optical switch input is sequentially connected to each of a selected group of said third optical switch plurality of outputs.

49. The medical apparatus of claim 46 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said third optical switch to connect said third optical switch input to a selected one of said third optical switch plurality of outputs;
cycling said first optical switch such that each of a selected group of said first optical switch plurality of inputs are sequentially connected to said first optical switch output.

50. The medical apparatus of claim 46 wherein said plurality of optical source inputs is optically isolated from said reflections.

51. The medical apparatus of claim 46 wherein said optical junction is an optical circulator configured to optically connect said plurality of optical source inputs with said plurality of diagnostic optical fibers in a first direction and optically isolate said plurality of optical source inputs from said plurality of diagnostic optical fibers in an opposite direction, to optically connect said optical detector output with said plurality of diagnostic optical fibers, and to optically isolate said optical detector output from said plurality of optical source inputs.

52. The medical apparatus of claim 46 further comprising:
a treatment optical fiber carried by said catheter; and
a treatment laser optically connected to said treatment optical fiber, wherein said first optical switch optically isolates said plurality of optical source inputs from said plurality of diagnostic optical fibers during operation of said treatment laser.

53. The medical apparatus of claim 46 further comprising a conduit defined by said catheter, said at conduit adapted for a procedure selected from the group consisting of fluid removal, angioplasty balloon insertion, angioplasty balloon inflation, and stent insertion.

54. The medical apparatus of claim 46 further comprising a plurality of sources each producing light of a selected wavelength, each of said plurality of sources connected to one of said plurality of optical source inputs.

55. A medical apparatus for safely navigating a lumen using a catheter, which can also differentiate between various objects found within the lumen, and for treating various conditions existing within the lumen, said medical apparatus comprising:
a catheter;
a plurality of diagnostic optical fibers carried by said catheter;
an optical source input producing light having a selected wavelength;
a plurality of optical detector outputs;
an optical junction in optical communication with said optical source input; and
a first optical switch in optical communication between said plurality of optical detector outputs and said optical junction, said first optical switch optically connecting one of said plurality of optical detector outputs to said optical junction so as to receive reflectances introduced in said diagnostic optical fiber subsequent to activation of said optical source input; and
a second optical switch in optical communication between said optical junction and said plurality of diagnostic optical fibers, said second optical switch optically connecting a selected one of said plurality of diagnostic optical fibers to said optical junction.

56. The medical apparatus of claim 55 further comprising a controller providing position awareness of and switching control over said first optical switch and said second optical switch.

57. The medical apparatus of claim 55 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said second optical switch such that said second optical switch input is connected to a selected one of said second optical switch plurality of outputs;
cycling said third optical switch such that said third optical switch input is sequentially connected to each of a selected group of said third optical switch plurality of outputs.

58. The medical apparatus of claim 55 further comprising a controller in communication with said first optical switch and said second optical switch, wherein said first optical switch has a plurality of inputs and an output and wherein said second optical switch has an input and a plurality of outputs, said controller performing a method for sequencing the operation of said medical apparatus, said method comprising the steps of:
operating said third optical switch to connect said third optical switch input to a selected one of said third optical switch plurality of outputs;
cycling said second optical switch such that said second optical switch input is sequentially connected to each of a selected group of said second optical switch plurality of outputs.

59. The medical apparatus of claim 55 wherein said optical source input is optically isolated from said reflectances.

60. The medical apparatus of claim 55 wherein said optical junction is an optical circulator configured to optically connect said optical source input with said plurality of diagnostic optical fibers in a first direction and optically isolate said optical source input from said plurality of diagnostic optical fibers in an opposite direction, to optically connect said plurality of optical detector outputs with said plurality of diagnostic optical fibers, and to optically isolate said plurality of optical detector outputs from said optical source input.

61. The medical apparatus of claim 55 further comprising:
a treatment optical fiber carried by said catheter; and
a treatment laser optically connected to said treatment optical fiber, wherein said first optical switch optically isolates said plurality of optical detector outputs from said plurality of diagnostic optical fibers during operation of said treatment laser.

62. The medical apparatus of claim 55 further comprising:
a treatment optical fiber carried by said catheter;
a treatment laser optically connected to said treatment optical fiber; and
an optical dead-end in communication with said first optical switch, said first optical switch optically connecting said optical dead-end and said optical junction during operation of said treatment laser.

63. The medical apparatus of claim 55 further comprising a conduit defined by said catheter, said at conduit adapted for a procedure selected from the group consisting of fluid removal, angioplasty balloon insertion, angioplasty balloon inflation, and stent insertion.

64. The medical apparatus of claim 55 further comprising a plurality of detectors each responsive to light of a selected wavelength, each of said plurality of detectors connected to one of said plurality of optical detector outputs.

65. A method for combining multiple techniques in a single catheter experience using a medical device having a plurality of optical inputs, a plurality of optical outputs, an optical junction, a plurality of optical fibers carried by a catheter, and a controller, said method comprising the steps of:
(a) connecting one of a plurality of optical inputs to an optical junction;
(b) connecting said optical junction to one of a plurality of optical outputs; and
(c) sequentially connecting said optical junction to a each of a selected group of a plurality of optical fibers.

66. A medical apparatus for safely navigating a lumen using a catheter, which can also differentiate between various objects found within the lumen, and for treating various conditions existing within the lumen, said medical apparatus comprising:
means for receiving diagnostic illumination from a plurality of optical sources;
means for providing passage through a lumen;
plurality of means for carrying said diagnostic illumination and corresponding reflectances through said means for providing passage though a lumen;
means for distributing said reflectances from said means for carrying said diagnostic illumination and corresponding reflectances to a plurality of optical detectors;
means for injecting the diagnostic illumination from a selected one of the plurality of optical sources into said means for carrying said diagnostic illumination and corresponding reflectances;
means for injecting the reflectances from said means for carrying said diagnostic illumination and corresponding reflectances into a selected one of the plurality of detectors; and
means for routing the diagnostic illumination through and the reflectances from said means for carrying the diagnostic illumination and corresponding reflectances.

67. The medical apparatus of claim 66 further comprising means for controlling said means for injecting the diagnostic illumination, means for injecting the reflectances, and said means for routing.
